(19) United States
(12) Patent Application Publication
(10) Pub. No.: US 2026/0207966 A1
(43) Pub. Date: Jul. 23, 2026

(54) MACHINE LEARNING BASED OPTIMIZATION OF PHOTO-BIO-MODULATION DOSING

(71) Applicant: Alcon Inc., Fribourg (CH)

(72) Inventor: Paul R. Hallen, Colleyville, TX (US)

(21) Appl. No.: 19/398,776

(22) Filed: Nov. 24, 2025

Related U.S. Application Data
(60) Provisional application No. 63/748,010, filed on Jan. 22, 2025.

(51) Int. Cl. A61N 5/06; G06N 3/0475; G06T 7/00

(52) U.S. Cl. CPC A61N 5/0622; G06N 3/0475; G06T 7/0012; A61N 2005/0626; A61N 2005/0648; G06T 2207/20081; G06T 2207/30041

(57) ABSTRACT

A computer-implemented method of automated dosing optimization for photo-bio- modulation (PBM) treatment. Embodiments include a light source of a multi-spectral imaging device configured to generate outgoing light across a range of wavelengths, one or more filters of the multi-spectral imaging device configured to generate incoming light to allow only a subset of wavelengths from the range of wavelengths to pass through, and one or more sensors of the multi- spectral imaging device configured to detect the filtered incoming light. Embodiments include one or more processors configured to execute instructions that cause the system to generate, based on the detecting of the filtered incoming light, a set of multi-spectral images of an eye of a patient, and generate, based on the set of multi-spectral images and one or more attributes of the patient, a PBM dosing regimen for the patient.

### INTRODUCTION

Photo-bio-modulation (PBM) is a form of light therapy that utilizes non-ionizing forms of light sources such as lasers, light-emitting diodes (LEDs), and/or broadband light, in the visible and near infrared spectrum. In some cases, PBM therapy may be performed on a patient’s eye to treat certain ophthalmic diseases, such as retina disease. For example, a light source may be placed near or in contact with the eye, allowing the light energy (e.g., photons) to penetrate tissue and interact with chromophores in cells, resulting in photophysical and photochemical changes in diseased and damaged tissues. Such changes may accelerate wound healing and tissue regeneration, increase circulation, reduce acute inflammation, reduce pain, and/or help restore normal cellular function

PBM may be administered by a medical professional in an office and/or may be administered via a device that enables at-home treatment (e.g., a headset). The appropriate PBM dosing regimen for a particular patient may depend on a variety of factors related to a patient’s condition that are challenging to assess, and so in many cases a standardized dosing regimen may be used even in cases where different dosing regimens would produce better results and/or more optimal utilization of resources.

Accordingly, there is a need for improved techniques for optimizing PBM dosing.

### SUMMARY

In certain embodiments, one general aspect includes a computer-implemented method for automated dosing optimization for photo-bio-modulation (PBM) treatment. The computer-implemented method includes: generating, using a light source of a multi-spectral imaging device, outgoing light across a range of wavelengths; selectively filtering, using one or more filters of the multi-spectral imaging device, incoming light to allow only a subset of wavelengths from the range of wavelengths to pass through; detecting, using one or more sensors of the multi-spectral imaging device, the filtered incoming light; generating, based on the detecting, a set of multi-spectral images of an eye of a patient, each multi-spectral image of the set of multi-spectral images corresponding to a specific spectral band; generating, based on the set of multi-spectral images and one or more attributes of the patient, a PBM dosing regimen for the patient.

In certain embodiments, another general aspect includes a system. The system includes a memory having executable instructions and a processor in communication with the memory. The processor is configured to execute the instructions to perform the computer-implemented method for resource-efficient consent management described above.

In certain embodiments, another general aspect includes a computer-program product including a non-transitory computer-usable medium having computer-readable program code embodied therein. The computer-readable program code is adapted to be executed to implement the computer-implemented method for resource-efficient consent management described above.

### BRIEF DESCRIPTION OF THE DRAWINGS

FIG. 1 illustrates an example of a process for automated dosing optimization for photo-bio-modulation (PBM) treatment, in accordance with certain embodiments of the present disclosure.

FIG. 2 illustrates an example related to training a machine learning model for automated dosing optimization for PBM treatment, in accordance with certain embodiments of the present disclosure.

FIG. 3 illustrates an example of devices for capturing multi-spectral images (MSIs) and administering PBM treatment, in accordance with certain embodiments of the present disclosure.

FIGS. 4A and 4B illustrate examples of processes related to automated dosing optimization for PBM treatment, in accordance with certain embodiments of the present disclosure.

FIG. 5 illustrates an example of a computing device for automated dosing optimization for PBM treatment, in accordance with certain embodiments of the present disclosure.

### DETAILED DESCRIPTION

Recently, low light therapy such as photo-bio-modulation (PBM) for ophthalmic diseases has shown significant efficacy and safety. For example, low light therapy may be used to rejuvenate mitochondria in the retina by stimulation via specific wavelengths to treat deseases such as dry age-related macular disease (AMD), geographic atrophy, myopia, inherited retinal diseases, wet AMD, glaucoma, diabetic macular edema, diabetic retinopathy, and/or other degenerative eye diseases. An optimal dosing regimen for PBM to treat diseases of the eye may depend on a variety of factors related to a patient. For example, a patient’s medical history, personal characteristics, disease location and progression, and/or the like may impact the efficacy of different dosing regimens. Determining the precise characteristics of a patient’s disease may be challenging. Imaging techniques such as multi-spectral imaging may be used to identify biomarkers such as drusen and/or geographic atrophy, but it can be difficult for a healthcare professional to identify the precise quantity, locations, and sizes of such biomarkers in a multi-spectral image (MSI). Other types of ophthalmic testing (e.g., visual acuity, contrast sensitivity, dark adaptation, optical coherence tomography (OCT), micro-perimetry, scanning laser ophthalmoscopy, and/or the like) can also be used to determine the nature and extent of a patient’s condition, but it can be challenging to determine how particular test results impact optimal PDM dosing. Furthermore, determining the precise relationship between a given combination of patient attributes, including disease characteristics, and an outcome of a particular PBM dosing regimen is difficult to pinpoint.

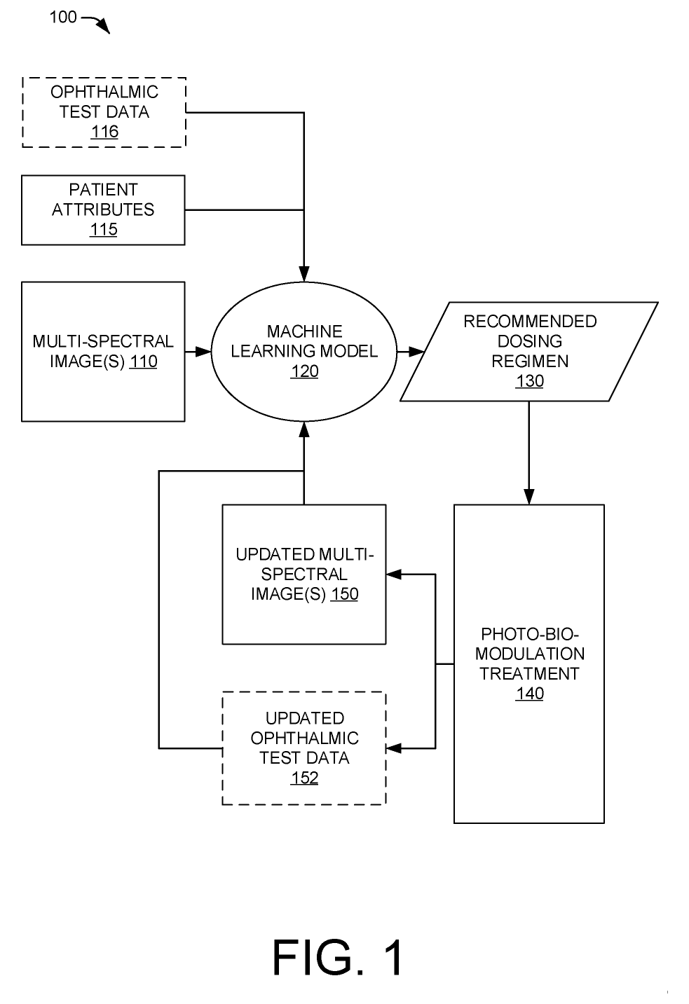

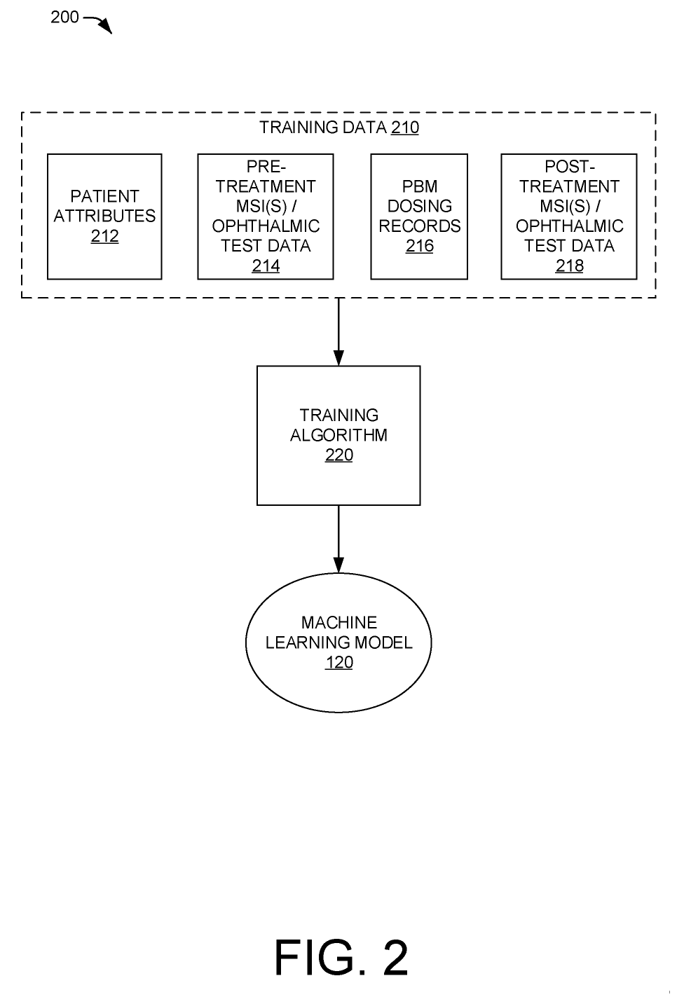

Aspects of the present disclosure overcome these challenges through the use of machine learning-based techniques for determining an optimal PBM dosing regimen for a given patient in an ophthalmic context based on attributes and condition information (e.g., including one or more MSIs and, in some embodiments, results of one or more ophthalmic tests) of the given patient, as described in more detail below with respect to FIG. 1. In certain embodiments, records of PBM treatments of a plurality of patients associated with patient attributes and MSIs (and, in some embodiments, other ophthalmic test results) from before and after treatment (e.g., along with other records related to the patient’s condition) are used to generate training data for such a machine learning model. As described in more detail below with respect to FIG. 2, the machine learning model may learn which PBM dosing regimens produce the best outcomes for patients having particular attributes (e.g., including particular attributes indicated in MSIs and/or other ophthalmic test results) through a supervised learning process, such as based on a large dataset of patient information.

A machine learning model trained using techniques described herein may be used to determine a recommended quantity and frequency of PBM treatment for a particular patient, and the recommended quantity and frequency may be used to administer PBM to the particular patient, such as a via a headset. The patient’s condition may be tracked after administering PBM, such as by capturing one or more updated MSIs of the patient and/or performing one or more ophthalmic tests on the patient to determine updated test results, and the updated information about the patient’s condition may be used to provide updated inputs to the machine learning model in order to generate an updated recommended quantity and frequency of PBM treatment for the patient (e.g., in an ongoing feedback loop). Furthermore, the machine learning model may be retrained as new ground truth becomes available (e.g., based on updated MSIs and/or other ophthalmic test results after PBM treatment) for continuous improvement.

Embodiments of the present disclosure accomplish various technical improvements. For example, utilizing machine learning techniques to learn relationships between PBM dosing regimens and clinical outcomes for patients having particular attributes allows optimal PBM dosing regimens to be automatically determined in a dynamic, targeted, and accurate manner for individual patients. Furthermore, making use of MSI technology (and, in some embodiments, other ophthalmic tests) to capture and identify information related to the ophthalmic condition of a patient both prior to and after PBM treatment allows machine learning techniques described herein to be dynamically informed regarding the clinical effects of particular PBM dosing regimens in particular cases in a manner that is more accurate and objective than would be possible without the use of such imaging technology and/or other tests. Additionally, by determining optimal PBM dosing regimens that are targeted for a particular patient’s attributes and condition, techniques described herein avoid unnecessarily utilization of device resources in connection with performing PBM treatments that are unlikely to be beneficial to a given patient under a particular set of circumstances. Accordingly, the embodiments described herein improve utilization of resources, such as utilization of devices used in providing PBM treatments.

Embodiments of the present disclosure also involve interactive feedback loops by which the automated PBM dosing recommendation process is continuously improved based on updated information about a patient’s condition, such as generating updated recommendations and/or retraining a machine learning model based on updated MSIs and/or ophthalmic test results captured after administering PBM according to a dosing regimen determined using machine learning techniques described herein, thereby reducing suboptimal PBM dosing recommendations over time and improving the automated recommendation technology.

FIG. 1 illustrates an example of a process 100 for automated dosing optimization for photo-bio-modulation (PBM) treatment, in accordance with certain embodiments of the present disclosure.

In process 100, patient attributes 115, ophthalmic test data 116, and one or more multi-spectral images (MSIs) 110 are provided as inputs to a machine learning model 120. Machine learning model 120 may have been trained through a supervised learning process to output recommended photo-bio-modulation (PBM) dosing regimens for particular patients based on attributes, ophthalmic test results, and MSI(s) related to the patients. An example of such a training process is described below with respect to FIG. 2.

Machine learning model 120 may, for example, include a neural network, deep neural network (DNN), convolution neural network (CNN), recurrent neural network (RNN), region-based CNN (R-CNN), long short term memory (LSTM) model, autoencoder (AE) or other type of neural network, a tree-based model (e.g., random forest, gradient boosted tree model, and/or the like), a support vector machine, a generative adversarial network (GAN) model, a logistic regression model, and/or the like. In some embodiments, machine learning model 120 comprises a single model, while in other embodiments, machine learning model 120 comprises multiple models, such as an ensemble of models. In one particular implementation, machine learning model 120 comprises a computer vision model or layer such as a CNN for processing images (e.g., MSI(s) 110), such as outputting patient condition attributes extracted from such images, and an additional model or layer(s) that generates dosing recommendations based on outputs from the computer vision model and additional inputs (e.g., other patient attributes).

For example, machine learning model 120 may accept MSI(s) 110 as inputs, and may extract attributes from MSI(s) 110, such as the quantity, size, and location of particular biomarkers (e.g., drusen, geographic atrophy instances, and/or the like), and may use these extracted attributes along with patient attributes 115 (and, in some embodiments, other attributes indicated in ophthalmic test data 116) to generate recommended dosing regimen 130. Alternatively, machine learning model 120 may not explicitly extract any features from MSI(s) 110, and may simply use MSI(s) 110 themselves as features when generating recommended dosing regimen 130.

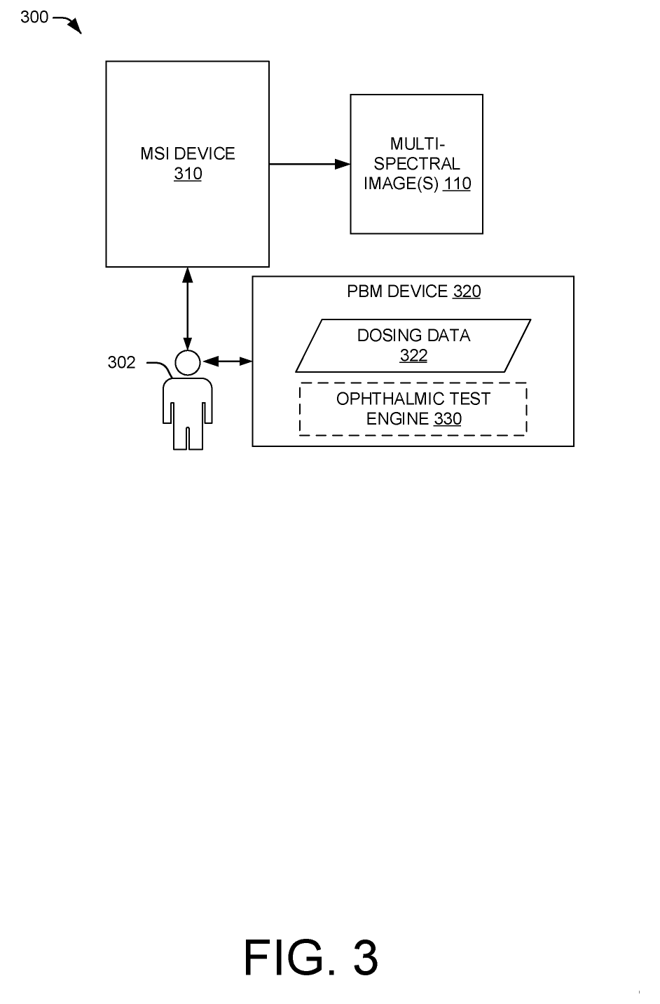

In some embodiments, MSI(s) 110 are captured using an MSI device such as a multi-spectral camera, as described below with respect to FIG. 3. For example, MSI(s) 110 may include one or more multi-spectral images of the eye of a patient that is associated with patient attributes 115. Multi-spectral imaging is an approach for imaging the eye within multiple wavelength bands. Typical MSI techniques illuminate the eye with narrow-band light at a plurality of different bands (e.g., across a range of wavelengths), and the light is generally detected by a detector after passing through one or more transmission filters matching the narrow-band light, such as to allow only a subset of wavelengths from the range of wavelengths to pass through. A set of MSIs may be generated based on such detection, each of which may correspond to a specific spectral band. MSIs enable the visualization of a wide array of retinal and choroidal pathologies including retinovascular disorders, retinal pigment epithelial changes, and choroidal lesions. For example, an MSI may include visible biomarkers such as drusen and geographic atrophy that are not visible in other types of images. An MSI cannot be captured or processed mentally, as capturing and processing an MSI involves generating light at multiple different wavelengths, passing the light through one or more particular transmission filters, and detecting the light after such filtering, none of which can be performed in the human mind.

Patient attributes 115 generally includes information about a patient, such as personal characteristics (e.g., age, gender, national origin, and/or the like), medical history (e.g., known medical conditions, information about the extent of known medical conditions, procedures that have been performed on the patient, medications taken by the patient, information about medical conditions of family members, and/or the like), and/or other information about the patient and/or the patient’s medical condition. In some embodiments, patient attributes 115 includes information about past PBM treatments performed on the patient, such as dosing information (e.g., quantity and frequency of treatments) and/or when (e.g., how recently) such treatment(s) were performed.

Ophthalmic test data 116 generally includes results of performing one or more ophthalmic tests, such as visual acuity, contrast sensitivity, dark adaptation, optical coherence tomography (OCT), micro-perimetry, scanning laser ophthalmoscopy, and/or the like, on the patient. For example, ophthalmic test data 116 may include one or more scores, pass or fail indicators, and/or other values indicating results of such testing, and may generally provide insight into the patient’s ophthalmic condition.

Machine learning model 120 processes patient attributes 115, ophthalmic test results 116, and MSI(s) 110 (or some subset of these, or features derived from these) and, in response, outputs recommended dosing regimen 130. Recommended dosing regimen 130 may indicate an amount and/or frequency of PBM treatment recommended for the patient to achieve an optimal result (e.g., to achieve a maximum improvement and/or reduction in deterioration in the patient’s condition). One example of a recommended dosing regimen 130 includes dosing of three wavelengths in nine treatments over a period of five weeks, such as including 40 second treatments at 0.4 Joules per square centimeter (J/cm²) each.

PBM treatment 140 may be performed based on recommended dosing regimen 130. For example, as described in more detail below with respect to FIG. 3, a PBM device (e.g., headset) may be used to administer one or more doses of PBM to the patient according to recommended dosing regimen 130. In some embodiments, a PBM device is programmed to administer PBM treatment 140 according to particular parameters specified in PBM treatment 140. In some embodiments, PBM treatment 140 involves Yttrium Aluminum Garnett (YAG) laser technology, femtosecond laser technology, and/or picosecond laser technology. PBM treatment 140 may involve generating light across one or more wavelengths such as 590 nanometers, 660 nanometers, or 850 nanometers.

PBM treatment 140 may be performed via one or more of light sources, such as including an LED configured to emit light at one or more wavelengths that stimulate cytochrome –C-Oxidase and/or the Adenosine triphosphate (ATP) energy cycle, such as to improve retina function and/or health. Studies have shown that particular wavelengths, such as 590 nanometers, 660 nanometers, and 850 nanometers, can improve vision by an average of six letters. Light sources may include one or more lasers configured to heat the retinal pigment epithelium (RPE) and create heat shock proteins to improve RPE metabolism and reduce both oxidative stress and inflammation. Such therapy can improve the permeability of Bruchs membrane, reduce thickening, and ultimately redice the size and/or number of drusen deposits (which are a hallmark of retina dysfunction). Such heat shock proteins have been shown to be effective in other diseases, establishing proof of concept for retina use. The laser technology could be Yttrium Aluminum Garnett (YAG) laser technology, frequency double YAG laser technology, femtosecond laser technology, and/or picosecond laser technology. Laser device delivery can be continuous, pulse, or micro-pulse, etc., such as depending on the stage of disease. Further, laser(s), LED(s), and/or superluminescent LED(s) used in PBM treatment 140 may perform low power therapy for metabolic retina health and/or heat shock protein therapy.

After PBM treatment 140 (e.g., after one or more of the doses recommended in recommended dosing regimen 130 have been completed), one or more updated MSIs 150 may be captured, such as in a similar manner to that in which MSI(s) 110 were captured (e.g., using an MSI device). Updated MSI(s) may indicate any changes in the patient’s condition (or a lack of change in the patient’s condition) that resulted from the PBM treatment 140. For example, the quantity, location, and/or size of one or more biomarkers may have changed between MSI(s) 110 and updated MSI(s) 150, and such a change may indicate a change in the patient’s condition resulting from PBM treatment 140. If there is no change in such biomarkers between MSI(s) 110 and updated MSI(s) 150, this may indicate that the patient’s condition did not change as a result of PBM treatment 140.

Also, after PBM treatment 140 (e.g., after one or more of the doses recommended in recommended dosing regimen 130 have been completed), updated ophthalmic test data 152 may be generated, such as in a similar manner to how ophthalmic test data 116 was generated. For example, one or more ophthalmic tests may be performed on the patient (e.g., using the same device used to administer PBM and/or one or more different devices) to determine any effect of PBM treatment 140 on the patient’s condition.

Updated MSI(s) 120 and/or updated ophthalmic test data 152 may be provided to machine learning model 120 along with patient attributes 115 (e.g., which may also be updated to include one or more updated attributes, such as indicating a change in the patient’s condition, treatment history, and/or the like, such as a change in a patient’s visual acuity or contrast) in order to determine an updated recommended dosing regimen. For example, machine learning model 120 may output an updated recommended dosing regimen in response to updated MSI(s) 150, updated ophthalmic test data 152, and/or patient attributes 115 (which may also be updated), such as indicating a different quantity and/or frequency of PBM. The updated recommended dosing regimen may be used to perform further PBM treatment(s), after which additional updated MSI(s) and/or ophthalmic test data may be captured and used to generate a further updated recommended dosing regimen (e.g., in a feedback loop).

In some embodiments, machine learning model 120 may be re-trained based on updated MSI(s) 150, updated ophthalmic test data 152, and/or one or more updated patient attributes. For example, updated training data may be generated based on updated MSI(s) 150, updated ophthalmic test data 152, and/or one or more updated patient attributes (e.g., indicating a change in the patient’s condition) such that machine learning model 120 is able to learn from the actual outcome of performing PBM treatment 140 for the patient according to recommended dosing regimen 130. Once re-trained, machine learning model 120 may be used to generate subsequent recommended dosing regimens for the same patient or different patients with a higher level of accuracy.

FIG. 2 illustrates an example 200 related to training a machine learning model for automated dosing optimization for PBM treatment, in accordance with certain embodiments of the present disclosure. Example 200 includes machine learning model 120 of FIG. 1.

In example 200, training data 210 is used by a training algorithm 220 to train machine learning model 120 through a supervised learning process. Training data 210 includes (or is based on) patient attributes 212, pre-treatment MSI(s) and/or ophthalmic test data 214, PBM dosing records 216, and post-treatment MSI(s) and/or ophthalmic test data 218. Patient attributes 212 generally include attributes of one or more patients, such as personal traits, medical history, and/or the like. Pre-treatment MSI(s) and/or ophthalmic test data generally include MSI(s) and/or other ophthalmic test results of the one or more patients captured before PBM treatment was performed, while post-treatment MSI(s) 218 generally include MSI(s) and/or other ophthalmic test results of the one or more patients captured after PBM treatment was performed. PBM dosing records 216 generally includes records of PBM treatments that were administered to the one or more patients according to particular dosing regimens.

Generally, training data 210 includes data indicating how particular dosing regimens (indicated in PBM dosing records 216) impacted the condition (e.g., as represented by changes between pre-treatment MSI(s) and/or ophthalmic test data 214 and post-treatment MSI(s) and/or ophthalmic test data 218 and/or by other data indicating changes in condition, such as in patient attributes 212) of one or more patients associated with patient attributes 212.

Training algorithm 220 generally utilizes training data 210 to train machine learning model 120. For example, training algorithm 220 may involve associating sets of input features (e.g., subsets of patient attributes 212 and pre-treatment MSI(s) and/or ophthalmic test data 214) with labels indicating dosing regimens (e.g., indicated in PBM dosing records 216) that resulted in positive outcomes (e.g., as indicated by differences or lack of difference between pre-treatment MSI(s) and/or ophthalmic test data 214 and post-treatment MSI(s) and/or ophthalmic test data 218 and/or other differences or lack of difference in attributes) and/or, in some embodiments, dosing regimens that resulted in negative outcomes (e.g., as indicated by differences or lack of difference between pre-treatment MSI(s) and/or ophthalmic test data 214 and post-treatment MSI(s) and/or ophthalmic test data 218 and/or other differences or lack of difference in attributes) for those patients. In some cases, an image processing model, such as a computer vision model, is used as part of training algorithm 220 to analyze pre-treatment MSI(s) and/or ophthalmic test data 214 and post-treatment MSI(s) and/or ophthalmic test data 218 and extract information about a patient’s condition from such images, such as the quantity, location, and size of certain biomarkers, and this extracted information is used by training algorithm 220 to determine which dosing regimens resulted in positive outcomes for particular patients and which dosing regimens resulted in negative outcomes for certain patients.

In some embodiments, labeled training data such as including sets of input features (e.g., subsets of patient attributes 212 and pre-treatment MSI(s) and/or ophthalmic test data 214) labeled with dosing regimens that resulted in positive outcomes (e.g., as positive training examples) and/or labeled with dosing regimens that resulted in negative outcomes (e.g., as negative training examples) is used in a supervised learning process to train machine learning model 120. In a typical supervised learning process, a set of training inputs is provided to a model, the model generates an output in response to the set of training inputs, the generated output is compared to a label associated with the training inputs, and one or more parameters of the model are adjusted based on the comparing, such as iteratively until one or more conditions are met. For instance, the one or more conditions may relate to an objective function (e.g., a cost function), or may relate to whether the outputs produced by the model based on the training inputs match the labels associated with the training inputs or whether a measure of error between training iterations is not decreasing or not decreasing more than a threshold amount. The conditions may also include whether a training iteration limit has been reached. Parameters adjusted during training may include, for example, hyperparameters, values related to numbers of iterations, weights, functions used by nodes to calculate scores, and the like. In some embodiments, validation and testing are also performed for a machine learning model, such as based on validation data and test data, as is known in the art.

The training processes described above are included as examples, and other methods of training machine learning model 120 based on training data 210 are possible. In some embodiments, training algorithm 220 may involve one or more unsupervised learning processes (e.g., clustering), semi-supervised learning processes, and/or supervised learning processes. For example, unsupervised learning techniques or semi-supervised learning techniques may be used to analyze patient data in order to identify correlations between particular dosing regimens and positive outcomes for patients having particular attributes and/or pre-treatment MSI(s) and/or ophthalmic test data. The results of such unsupervised and/or semi-supervised learning techniques may then be used in a supervised learning process, such as labeling input features for use in supervised learning based on such results. In other embodiments, labeled training data for a supervised learning process may be generated based on manual analysis of patient information, including MSI(s) and/or ophthalmic test data, and/or based on manual confirmation of results of an unsupervised learning process. Labels may further be based on other indicators of outcomes of PBM dosing regimens, such as other notes and/or indicators of changes in a patient’s condition that may be included in patient attributes 212.

More generally, training algorithm 220 involves training machine learning model 120 based on training data 210 to recognize correlations between particular PBM dosing regimens and positive outcomes for patients having particular attributes (e.g., including particular pre-treatment MSI(s) and/or ophthalmic test data). It is understood that a variety of machine learning techniques exist for such a training process, and any suitable machine learning algorithm(s) and/or model(s) may be used to train machine learning model 120 to output recommended PBM dosing regimens based on input features indicating patient attributes and/or MSI(s) and/or ophthalmic test data.

Furthermore, training algorithm 220 may be used to re-train machine learning model 120 as new training data becomes available, such as when updated MSI(s) and/or ophthalmic test data of a patient are captured after administering PBM to the patient according to a recommended PBM dosing regimen that was generated using the trained machine learning model 120.

FIG. 3 illustrates an example 300 of devices for capturing multi-spectral images (MSIs) and administering PBM treatment, in accordance with certain embodiments of the present disclosure.

In example 300, an MSI device 310 is used to capture MSI(s) 110 of a patient 302. MSI device 310 may, for example, be a multi-spectral camera. In some embodiments, MSI device 310 captures the MSI(s) 110 by illuminating the eye of patient 302 using multi-spectral band illumination sources (e.g., narrowband illumination sources, narrowband filters, etc.) and/or measuring reflected light using multi-spectral band cameras (e.g., MSI device 310, which may comprise an imaging sensor capable of sensing multiple spectral bands, beyond RGB spectral bands). Accordingly, each image in MSI(s) 110 may represent reflected light within a specific spectral ban. Differences among images in MSI(s) 110 may result from different reflectivities of different structures within the eye for different spectral bands. MSI(s) 110, when considered collectively, therefore provide additional information about the structures of the eye than a single broadband image. In some implementations, MSI(s) 110 are en face images of the retina that are used to detect pathologies of the retina. However, MSI(s) 110 of other parts of the eye, such as the vitreous or anterior chamber may also be used with techniques described herein.

As described above, MSIs (e.g., MSI(s) 110) captured using MSI device 310 may be used, such as along with other ophthalmic test data and/or patient attributes, as part of a process for automatically determining a recommended PBM dosing regimen for patient 302. The recommended dosing regimen may then be used to administer PBM treatment to the patient 302 via a PBM device 320. For example, PBM device 320 may be configured with dosing data 322 that is based on such a recommended dosing regimen (e.g., recommended dosing regimen 130 of FIG. 1) in order to administer PBM treatment. Dosing data 322 may, for instance, indicate a quantity and/or frequency of PBM treatment.

PBM device 320 may, for example, be a wearable device (e.g., headset), handheld device, or other type of device comprising one or more light sources (e.g., lasers, LEDs, broadband light sources, and/or the like) that may be placed near or in contact with a patient’s eye or eyelid in order to administer PBM treatment. PBM device 320 may be configured to recognize a particular patient 302, such as based on or more credentials and/or biological characteristics (e.g., based on a retinal scan), and, if the patient 302 is recognized, to administer an appropriate PBM dose to that particular patient 302 according to dosing data 322, such as limiting treatments to prescribed lengths, types, and/or frequencies (e.g., only permitting treatment at particular intervals, at set times, and/or otherwise as specified in dosing data 322). In some embodiments, PBM device 320 is further configured to generate notifications, such as sending a notification to a separate device (e.g., a mobile phone) associated with patient 302 when PBM treatment is due for patient 302.

PBM device 320 may also include an ophthalmic test engine 330, which may be configured to administer one or more ophthalmic tests to the patient, such as before and/or after administering PBM treatment, such as using hardware of PBM device 320. For example, such tests may include visual acuity, contrast sensitivity, dark adaptation, optical coherence tomography (OCT), micro-perimetry, scanning laser ophthalmoscopy, and/or the like. Alternatively or additionally, ophthalmic testing may be performed using one or more different devices than PBM device 320.

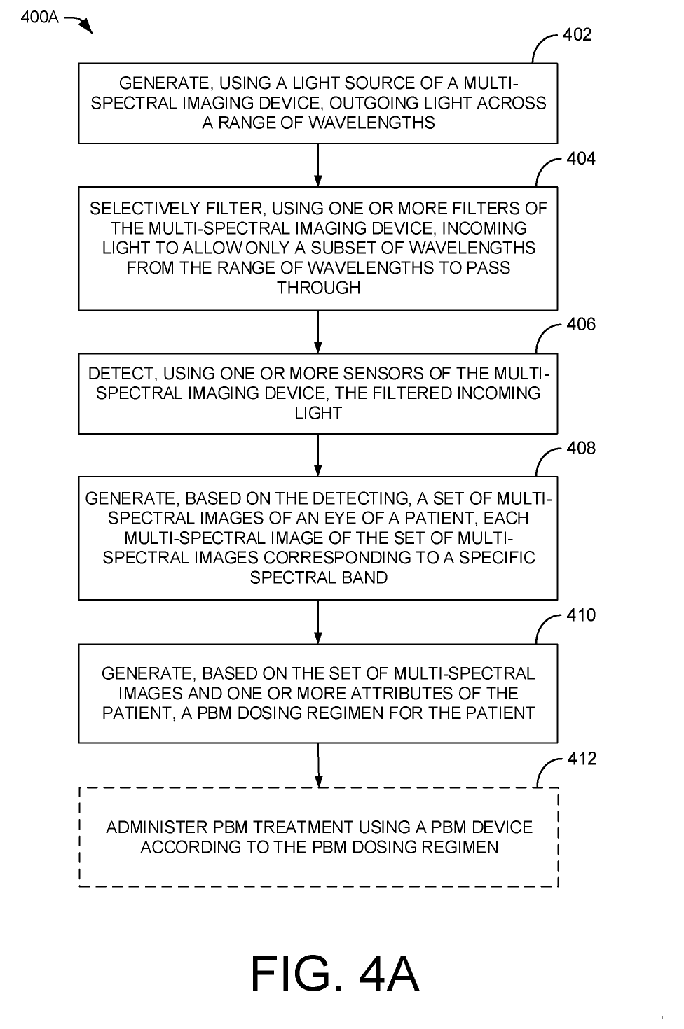

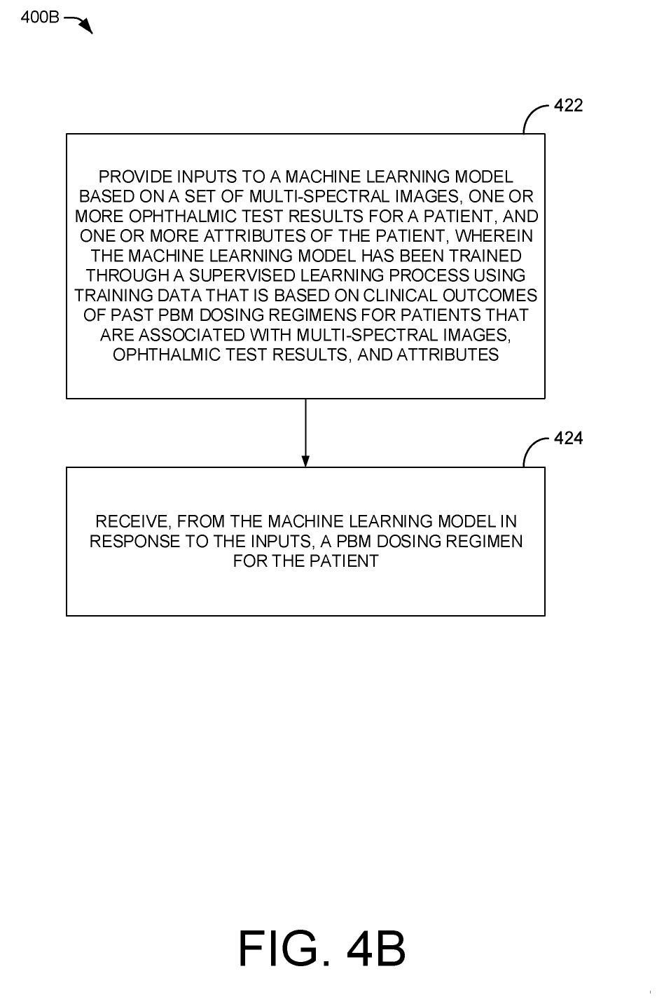

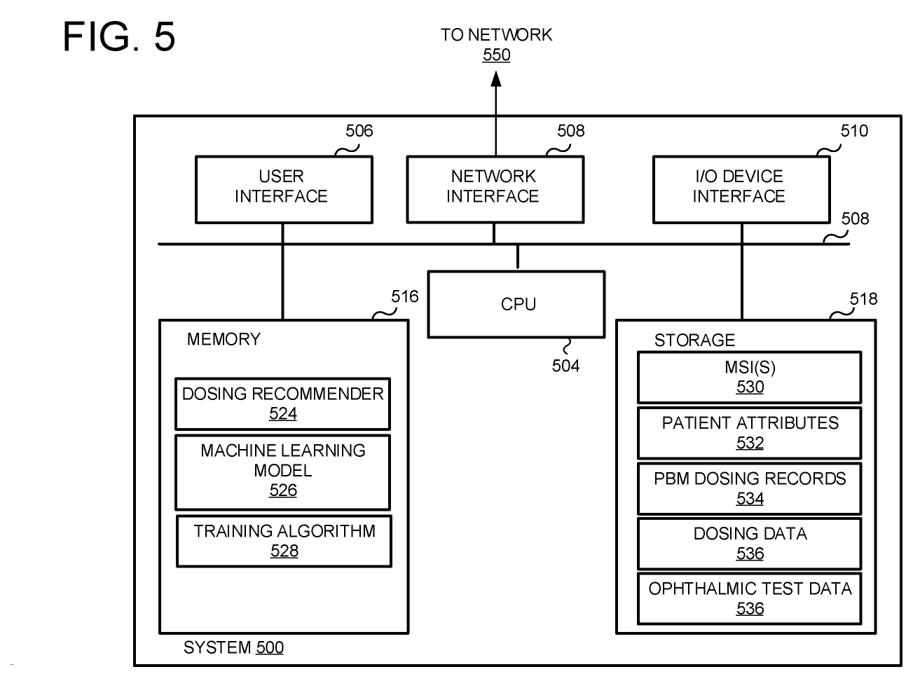

FIG. 4A illustrates an example of a process 400A for automated dosing optimization for PBM treatment, in accordance with certain embodiments of the present disclosure. In certain embodiments, the process 400A can be implemented by one or more components described above with respect to FIGS. 1-3 and/or below with respect to FIG. 5. It is noted that any number of systems, in whole or in part, can implement the process 400A.

Process 400A begins at block 402, with generating, using a light source of a multi-spectral imaging device, outgoing light across a range of wavelengths.

Process 400A continues at block 404, with selectively filtering, using one or more filters of the multi-spectral imaging device, incoming light to allow only a subset of wavelengths from the range of wavelengths to pass through.

Process 400A continues at block 406, with detecting, using one or more sensors of the multi-spectral imaging device, the filtered incoming light.

Process 400A continues at block 408, with generating, based on the detecting, a set of multi-spectral images of an eye of a patient, each multi-spectral image of the set of multi-spectral images corresponding to a specific spectral band.

Process 400A continues at block 410, with generating, based on the set of multi-spectral images and one or more attributes of the patient, a PBM dosing regimen for the patient. In some embodiments, the generating of the PBM dosing regimen for the patient is based further on results of performing one or more ophthalmic tests on the patient, such as visual acuity, contrast sensitivity, dark adaptation, optical coherence tomography (OCT), micro-perimetry, scanning laser ophthalmoscopy, and/or the like.

In some embodiments, generating the PBM dosing regimen comprises providing inputs to a machine learning model based on the set of multi-spectral images and the one or more attributes of the patient, wherein the machine learning model has been trained through a supervised learning process using training data that is based on clinical outcomes of past PBM dosing regimens for patients that are associated with multi-spectral images and attributes (and, in some embodiments, ophthalmic test results), and receiving, from the machine learning model in response to the inputs, the PBM dosing regimen for the patient, wherein one or more PBM treatments are administered to the patient according to the PBM dosing regimen.

Process 400A optionally continues at block 412, with administering PBM treatment using a PBM device according to the PBM dosing regimen for the patient.

In certain embodiments, the PBM dosing regimen comprises a dosing frequency and a dosing amount.

In some embodiments, the machine learning model analyzes one or more biomarkers that are included in the set of multi-spectral images in order to generate the PBM dosing regimen.

In certain embodiments, the machine learning model analyzes a quantity, a location, and a size of each of the one or more biomarkers that are included in the set of multi-spectral images in order to generate the PBM dosing regimen. In some embodiments, the one or more biomarkers comprise one or more of: a drusen body; or an instance of geographic atrophy.

Some embodiments further comprise generating one or more updated multi-spectral images of the eye of the patient after one or more PBM treatments are administered to the patient according to the PBM dosing regimen, and generating an updated PBM dosing regimen for the patient based on the one or more updated multi-spectral images.

In certain embodiments, a machine learning model is re-trained using updated training data that is based on the one or more updated multi-spectral images.

FIG. 4A illustrates an example of a process 400B for automated dosing optimization for PBM treatment, in accordance with certain embodiments of the present disclosure. In certain embodiments, the process 400B can be implemented by one or more components described above with respect to FIGS. 1-3 and/or below with respect to FIG. 5. It is noted that any number of systems, in whole or in part, can implement the process 400B. In some embodiments, process 400B represents further details of step 410 of process of 400A of FIG. 4A.

Process 400B begins at block 422, with providing inputs to a machine learning model based on a set of multi-spectral images (e.g., generated at block 408 of FIG. 4A), one or more ophthalmic test results for a patient, and one or more attributes of the patient, wherein the machine learning model has been trained through a supervised learning process using training data that is based on clinical outcomes of past PBM dosing regimens for patients that are associated with multi-spectral images, ophthalmic test results, and attributes.

In certain embodiments, the one or more attributes of the patient comprise one or more of: a personal trait; or a medical history.

In some embodiments, the one or more ophthalmic test results for the patient comprise one or more of: visual acuity test results, contrast sensitivity test results, dark adaptation test results, optical coherence tomography (OCT) test results, micro-perimetry test results, or scanning laser ophthalmoscopy test results.

Process 400B continues at block 424, with receiving, from the machine learning model in response to the inputs, a PBM dosing regimen for the patient, wherein one or more PBM treatments are administered to the patient according to the PBM dosing regimen.

FIG. 5 illustrates an example of a system 500 for automated dosing optimization for PBM treatment, in accordance with certain embodiments of the present disclosure. For example, system 500 may be configured to perform method 400 of FIG. 4 and/or other aspects of the present disclosure, such as discussed above with respect to FIGS. 1-3.

As shown, system 500 includes, without limitation, central processing unit (CPU) 504, user interface 506, network interface 508, memory 516, storage 518, interconnect 508, and at least one I/O device interface 510 which may allow for the connection of various I/O devices (e.g., keyboards, displays, mouse devices, pen input, etc.) to system 500. While one or more operations are described herein as being performed by particular components of system 500, those operations may, in some embodiments, be performed by other components of system 500 and/or component(s) of other system(s). As an example, while one or more operations are described herein as being performed by CPU 505, memory 516, and/or storage 518 those operations may, in other embodiments, be performed by other components of system 500 or of a different system.

CPU 504 may be representative of one or more processing devices and/or cores. In some embodiments, CPU 504 may retrieve and execute programming instructions stored in memory 516. Similarly, CPU 504 may retrieve and store application data residing in memory 516. Interconnect 508 transmits programming instructions and application data, among CPU 504, I/O device interface 510, user interface 506, memory 516, storage 518, network interface 508, etc. In some embodiments, CPU 504 may correspond to a single CPU, multiple CPUs, or a single CPU having multiple processing cores. Additionally, in some embodiments, memory 516 represents volatile memory, such as random-access memory. In some embodiments, storage 518 may be non-volatile memory, such as a disk drive, solid state drive, or a collection of storage devices distributed across multiple storage systems.

System 500 can include a network interface 508 for connection with a data communications network (e.g., network 550), such as to communicate with other devices. The data communications network can be, or can include, one or more of a private network, a public network, a local or wide area network, the Internet, combinations of the same, and/or the like. The data communications network can include, for example, interfaces (e.g., application programming interfaces) for enabling interaction and communication between and among the components and systems of the computing environment (e.g., of FIG. 3) and/or other components and systems.

The memory 516 can include a dosing recommender 524, which generally represents a software application that performs functionality described herein related to automatically generating recommended PBM dosing regimens for patients. Dosing recommender 524 may make use of a machine learning model 516 that is also depicted in memory 516, such as to automatically generate PBM dosing recommendations. For example, machine learning model 516 may be representative of machine learning model 120 of FIGS. 1 and 2. Memory 516 further comprises a training algorithm 528, which may be representative of training algorithm 220 of FIG. 2 In other embodiments, machine learning model 526 may be trained on a separate system from the system (e.g., system 500) on which the trained model is used to generate recommended PBM dosing regimens.

The storage 518 can include MSI(s) 530, which may be representative of MSI(s) 110 of FIGS. 1 and 3 and/or pre-treatment MSI(s) 214 and/or post-treatment MSI(s) 218 of FIG. 2. . The storage 518 can further include patient attributes 532, which may be representative of patient attributes 115 of FIG. 1 and/or patient attributes 212 of FIG. 2. The storage 518 can further include PBM dosing records 534, which may be representative of PBM dosing records 216 of FIG. 2. The storage 518 can further include dosing data 536, which may be representative of recommended dosing regimen 130 of FIG. 1 and/or dosing data 322 of FIG. 3. The storage 518 can further include ophthalmic test data 536, which may be representative of ophthalmic test data 116 and/or updated ophthalmic test data 152 of FIG. 1 and/or pre-treatment ophthalmic test data 214 and/or post-treatment ophthalmic test data 218 of FIG. 2.

It is noted that system 500 is included as an example, and techniques described herein may be implemented via fewer or more components, either on the same or different devices, and devices may include physical and/or virtual devices.

As used herein, a phrase referring to “at least one of” a list of items refers to any combination of those items, including single members. As an example, “at least one of: a, b, or c” or “at least one of: a, b, and c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112(f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. A system for automated dosing optimization for photo-bio-modulation (PBM) treatment, the system comprising:
a light source of a multi-spectral imaging device configured to generate outgoing light across a range of wavelengths;
one or more filters of the multi-spectral imaging device configured to generate incoming light to allow only a subset of wavelengths from the range of wavelengths to pass through;
one or more sensors of the multi-spectral imaging device configured to detect the filtered incoming light; and
one or more processors configured to execute instructions that cause the system to:
generate, based on the detecting of the filtered incoming light, a set of multi-spectral images of an eye of a patient, each multi-spectral image of the set of multi-spectral images corresponding to a specific spectral band; and
generate, based on the set of multi-spectral images and one or more attributes of the patient, a PBM dosing regimen for the patient.

2. The system of claim 1, wherein the generating of the PBM dosing regimen comprises:
providing inputs to a machine learning model based on the set of multi-spectral images and the one or more attributes of the patient, wherein the machine learning model has been trained through a supervised learning process using training data that is based on clinical outcomes of past PBM dosing regimens for patients that are associated with multi-spectral images and attributes; and
receiving, from the machine learning model in response to the inputs, the PBM dosing regimen for the patient, wherein one or more PBM treatments are administered to the patient according to the PBM dosing regimen.

3. The system of claim 2, wherein the machine learning model analyzes one or more biomarkers that are included in the set of multi-spectral images in order to generate the PBM dosing regimen.

4. The system of claim 3, wherein the machine learning model analyzes a quantity, a location, and a size of each of the one or more biomarkers that are included in the set of multi-spectral images in order to generate the PBM dosing regimen.

5. The system of claim 3, wherein the one or more biomarkers comprise one or more of: a drusen body; or an instance of geographic atrophy.

6. The system of claim 1, wherein the generating of the PBM dosing regimen is further based on one or more ophthalmic test results for the patient.

7. The system of claim 6, wherein the one or more ophthalmic test results for the patient comprise results of one or more of:
a visual acuity test;
a dark adaptation test;
an optical coherence tomography (OCT) test;
a micro-perimetry test;
a multi-spectral imaging (MSI) test; or
a scanning laser ophthalmoscopy test.

8. The system of claim 1, wherein the one or more attributes of the patient comprise one or more of: a personal trait; or a medical history.

9. The system of claim 1, wherein the PBM dosing regimen comprises a dosing frequency and a dosing amount.

10. The system of claim 1, wherein the one or more processors are configured to execute additional instructions that cause the system to:
generate one or more updated multi-spectral images of the eye of the patient after one or more PBM treatments are administered to the patient according to the PBM dosing regimen; and
generate an updated PBM dosing regimen for the patient based on the one or more updated multi-spectral images.

11. The system of claim 10, wherein a machine learning model is re-trained using updated training data that is based on the one or more updated multi-spectral images.

12. A computer-implemented method of automated dosing optimization for photo-bio-modulation (PBM) treatment, the computer-implemented method comprising:
generating, using a light source of a multi-spectral imaging device, outgoing light across a range of wavelengths;
selectively filtering, using one or more filters of the multi-spectral imaging device, incoming light to allow only a subset of wavelengths from the range of wavelengths to pass through;
detecting, using one or more sensors of the multi-spectral imaging device, the filtered incoming light;
generating, based on the detecting, a set of multi-spectral images of an eye of a patient, each multi-spectral image of the set of multi-spectral images corresponding to a specific spectral band; and
generating, based on the set of multi-spectral images and one or more attributes of the patient, a PBM dosing regimen for the patient.

13. The computer-implemented method of claim 12, wherein the generating of the PBM dosing regimen comprises:
providing inputs to a machine learning model based on the set of multi-spectral images and the one or more attributes of the patient, wherein the machine learning model has been trained through a supervised learning process using training data that is based on clinical outcomes of past PBM dosing regimens for patients that are associated with multi-spectral images and attributes; and
receiving, from the machine learning model in response to the inputs, the PBM dosing regimen for the patient, wherein one or more PBM treatments are administered to the patient according to the PBM dosing regimen.

14. The computer-implemented method of claim 13, wherein the machine learning model analyzes one or more biomarkers that are included in the set of multi-spectral images in order to generate the PBM dosing regimen.

15. The computer-implemented method of claim 14, wherein the machine learning model analyzes a quantity, a location, and a size of each of the one or more biomarkers that are included in the set of multi-spectral images in order to generate the PBM dosing regimen.

16. The computer-implemented method of claim 14, wherein the one or more biomarkers comprise one or more of: a drusen body; or an instance of geographic atrophy.

17. The computer-implemented method of claim 12, wherein the one or more attributes of the patient comprise one or more of: a personal trait; or a medical history.

18. The computer-implemented method of claim 12, wherein the PBM dosing regimen comprises a dosing frequency and a dosing amount.

19. The computer-implemented method of claim 12, further comprising:
generating one or more updated multi-spectral images of the eye of the patient after one or more PBM treatments are administered to the patient according to the PBM dosing regimen; and
generating an updated PBM dosing regimen for the patient based on the one or more updated multi-spectral images.

20. A non-transitory computer-readable medium comprising instructions that, when executed via one or more processors of a computing system, cause the computing system to:
generate, using a light source of a multi-spectral imaging device, outgoing light across a range of wavelengths;
selectively filter, using one or more filters of the multi-spectral imaging device, incoming light to allow only a subset of wavelengths from the range of wavelengths to pass through;
detect, using one or more sensors of the multi-spectral imaging device, the filtered incoming light;
generate, based on the detecting, a set of multi-spectral images of an eye of a patient, each multi-spectral image of the set of multi-spectral images corresponding to a specific spectral band; and
generate, based on the set of multi-spectral images and one or more attributes of the patient, a photo-bio-modulation (PBM) dosing regimen for the patient.
